(19) United States
(12) Patent Application Publication
(10) Pub. No.: US 2019/0104927 A1
(43) Pub. Date: Apr. 11, 2019

(54) ENDOSCOPE

(71) Applicant: Olympus Corporation, Tokyo (JP)

(72) Inventor: Masahiro Mizuno, Aizuwakamatsu-shi (JP)

(73) Assignee: Olympus Corporation, Tokyo (JP)

(21) Appl. No.: 16/210,105

(22) Filed: Dec. 5, 2018

Related U.S. Application Data
(63) Continuation of application No. PCT/JP2017/008262, filed on Mar. 2, 2017.

(30) Foreign Application Priority Data
Jun. 9, 2016 (JP) 2016-115584

(51) Int. Cl. A61B 1/00; A61B 1/06

(52) U.S. Cl. CPC A61B 1/00096; A61B 1/00094; A61B 1/00121; A61B 1/00105; A61B 1/00131; A61B 1/00114; A61B 1/00163; A61B 1/0661

(57) ABSTRACT

An endoscope includes an insertion portion that extends in a longitudinal direction thereof and a channel is inserted from the user's hand side of the insertion portion into a distal end portion thereof. The distal end portion of the insertion portion includes a first facet orthogonal to the longitudinal direction. A second facet intersects with the first facet adjacent thereto. A third facet intersects the first facet and the second facet adjacent thereto. An opening defined in the distal end portion side of the channel has a projected plane disposed in a position including a point where three boundaries between the first through third facets are intersect with one another.

### CROSS-REFERENCE TO RELATED APPLICATIONS

This application is a continuation application of PCT Application No. PCT/JP2017/008262 filed on Mar. 2, 2017, which in turn claim priority to the Japanese Patent Application No. 2016-115584 filed on Jun. 9, 2016 in Japan which is hereby incorporated by reference in its entirety.

### TECHNICAL FIELD

The technology disclosed herein generally relates to an endoscope having an objective lens, an illuminating lens, and a lumen opening on a distal end face of an insertion portion thereof.

### DESCRIPTION OF THE RELATED ART

Endoscopes have an objective lens and an illuminating lens arrayed on a distal end face of an insertion portion thereof. One type of endoscopes for medical use is referred to as a biliopancreatic endoscope whose insertion portion is pushed into a narrow lumen to insert the distal end of the insertion portion deeply into the lumen.

Japanese Patent Laid-Open No. 2012-75658 discloses an endoscope device that aims at (i) inserting the distal end of an endoscope insertion portion smoothly into a small-diameter biliopancreatic duct, (ii) increasing the visibility of an observation field of view, and (iii) allowing surgical techniques using treatment tools to be observed with ease. The distal end face of the endoscope insertion portion disclosed in the aforementioned document has an observation window, an illumination window, and a forceps port disposed thereon. The observation window projects on an axially distal end of the endoscope insertion portion. The distal end face extends from the observation window toward an outer circumferential surface of the endoscope insertion portion and includes a tapered slanting surface. The forceps port is opened in the slanting surface, and the illumination window is disposed on the slanting surface at an intermediate position between the observation window and the forceps port. As a result, the endoscope insertion portion has a tapered slanting surface extending fully circumferentially from the observation window toward the outer circumferential surface of the endoscope insertion portion, so that the distal end side of the endoscope insertion portion has a tapered shape.

### BRIEF SUMMARY OF EMBODIMENTS

The technology disclosed herein is directed to an endoscope that prevents the mucous membrane from being suctioned thereto in a suction process and which is of excellent manipulability for pushing an endoscope insertion portion smoothly into a narrow lumen.

An endoscope according to the present disclosure includes an insertion portion that extends in a longitudinal direction thereof and a channel is inserted from the user's hand side of the insertion portion into a distal end portion thereof. The distal end portion of the insertion portion includes a first facet orthogonal to the longitudinal direction. A second facet intersects with the first facet adjacent thereto and a third facet intersects the first facet and the second facet adjacent thereto. An opening defined in the distal end portion side of the channel has a projected plane disposed in a position including a point where three boundaries between the first through third facets intersect with each other.

One aspect of the disclosed technology is directed to an endoscope comprises an insertion portion that extends in a longitudinal direction thereof. A first facet is disposed on a distal end portion of the insertion portion and is positioned orthogonally to the longitudinal direction. A second facet is disposed on the distal end portion of the insertion portion and is intersected with the first facet adjacent thereto. A third facet is disposed on the distal end portion of the insertion portion and is intersected with the first facet and the second facet adjacent thereto. A fourth facet is disposed on the distal end portion of the insertion portion and is intersected with the first facet and the third facet adjacent thereto. A first opening is held in fluid communication with a distal end of a first channel inserted in the insertion portion in which a projected plane of the first opening at the distal end portion of the insertion portion is disposed in a position including a point where three boundaries between the first facet, the second facet, and the third facet intersect with each other. A second opening is held in fluid communication with a distal end of a second channel inserted in the insertion portion in which a projected plane of the second opening at the distal end portion of the insertion portion is disposed in a position including a boundary across which the first facet and the fourth facet intersect with each other.

Another aspect of the disclosed technology is directed an endoscope comprises an insertion portion having a distal end portion that extends in a longitudinal direction. A first channel and a second channel each of which is inserted through the insertion portion. The distal end portion includes a first facet is positioned orthogonally to the longitudinal direction. A second facet is intersected with the first facet adjacent thereto. A third facet is intersected with the first facet and the second facet adjacent thereto. A fourth facet is intersected with the first facet and the third facet adjacent thereto. A first opening is held in fluid communication with a distal end of the first channel in which a projected plane of the first opening at the distal end portion is disposed in a position including a point where three boundaries between the first facet, the second facet, and the third facet are intersected with one another. A second opening is held in fluid communication with a distal end of the second channel in which a projected plane of the second opening at the distal end portion is disposed in a position including a boundary across which the first facet and the fourth facet are intersected with one another.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view illustrating the manner in which an endoscope insertion portion commonly can be pushed and inserted into a narrow lumen.

FIG. 2 is a perspective view of an endoscope according to the present disclosure described herein.

FIG. 3 is a view illustrating a channel that is used as a suction channel and a treatment tool insertion channel of the endoscope.

FIG. 4A is a fragmentary perspective view of a distal end side of an insertion portion of the endoscope.

FIG. 4B is a front elevational view of the distal end side of the insertion portion of the endoscope.

FIG. 4C is a side elevational view of the distal end side of the insertion portion depicted in FIG. 4B as viewed from the direction indicated by the arrow Y4C.

FIG. 5 is a view illustrating the relationship between a first facet, a second facet, a third facet, a fourth facet, and a plurality of through-holes on a distal end face.

FIG. 6A is a view illustrating an example of operation of the distal end face of the distal end.

FIG. 6B is a view illustrating another example of operation of the distal end face of the distal end.

FIG. 6C is a view illustrating still another example of operation of the distal end face of the distal end.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

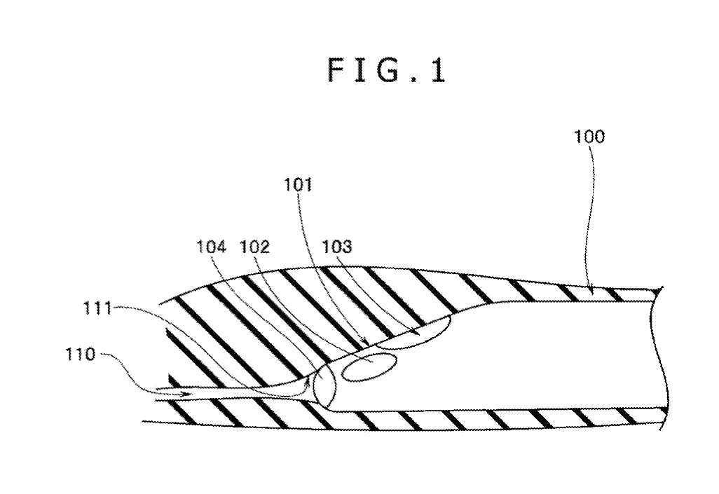

According to the endoscope insertion portion of the endoscope device, the observation window is positioned on the axially distal end of the endoscope insertion portion, and the illumination window and the forceps opening are disposed on the slanting surface. Therefore, when an endoscope insertion portion 100 is inserted into a narrow lumen 110 as depicted in FIG. 1, an illumination window 102 and a forceps port opening 103 that are disposed on a slanting surface 101 may be covered by a wall 111 of the lumen 110 that is spread. If the observation window 104 is not covered by the wall 111, an endoscopic image of the inside of the lumen 110 is displayed on an observation screen. Therefore, the user can observe the inside of the lumen 110. However, it is difficult for the user to determine that the forceps port opening 103 is covered by the wall 111 from the displayed endoscopic image. Accordingly, when the user starts a suction process to draw dirt and body fluids displayed on the observation screen from the lumen 110 using the endoscope insertion portion 100, the mucous membrane of the wall 111 may be suctioned to the forceps port opening 103, preventing dirt and body fluids from being drawn.

An embodiment of the present invention will be described hereinafter with reference to the drawings. In each of the figures used in the description that follows, some of the components are drawn to different scales in order to illustrate themselves in sizes large enough to be recognized in the figures. The present disclosure should not be limited to the numbers, shapes, size proportions, and relative positional relationships of the components depicted in the figures.

FIG. 1 is a view illustrating the manner in which an endoscope insertion portion commonly can be pushed and inserted into a narrow lumen. FIG. 1 is presented to facilitate an understanding of the state of the art and the differences the present disclosure provides with respect to the current practice depicted by FIG. 1.

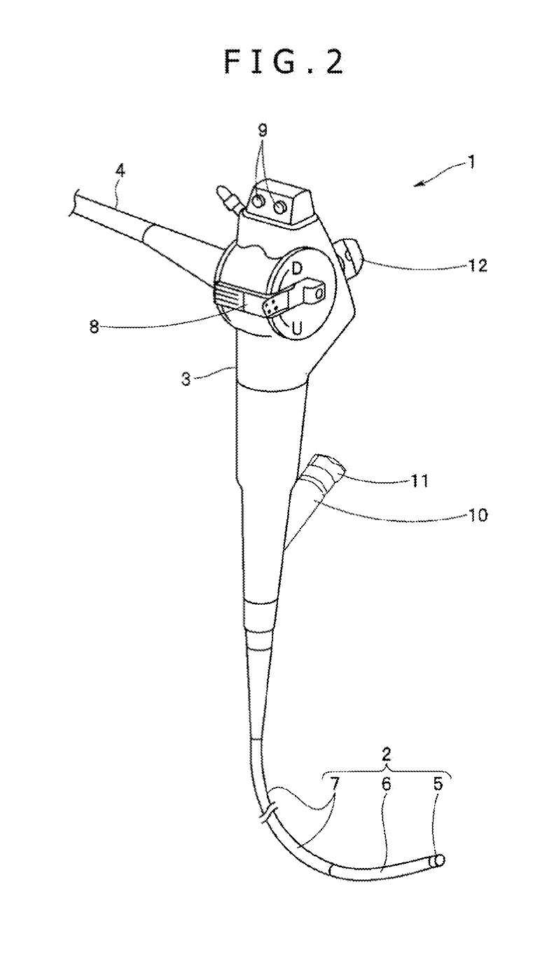

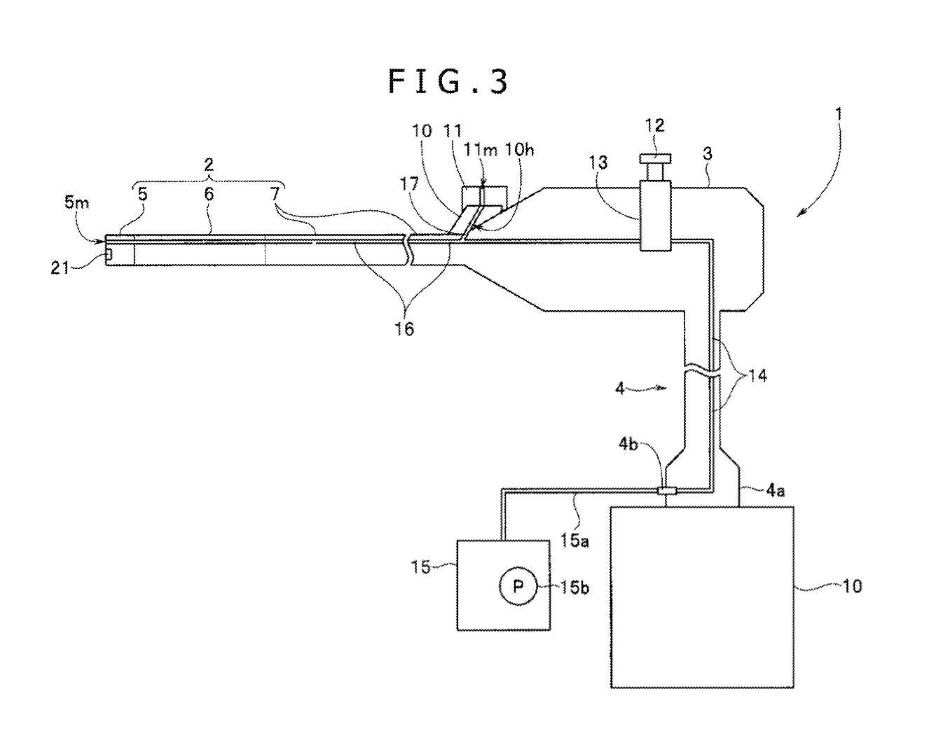

As depicted in FIG. 2, an endoscope 1 includes an insertion portion 2, an operation portion 3, and a universal cord 4. The insertion portion 2 extends in longitudinal direction thereof and is constructed of tubular or slender shape and is made of a flexible material. The operation portion 3 includes a grip joined to a proximal end side of the insertion portion 2. The universal cord 4 extends from a side of the operation portion 3. The universal cord 4 has on one end thereof an endoscope connector 4a for connecting to a light source 10 in FIG. 3.

The insertion portion 2 has a distal end portion 5, a bendable portion 6 that is bendable, and a pliable flexible tube 7, which are joined together successively from a distal end side of the insertion portion 2. The operation portion 3 has a bend operation lever 8 and remote switches 9 disposed thereon. The remote switches 9 are switches for giving image control instructions such as freeze, release, etc. The bendable portion 6 is bent as the bend operation lever 8 is turned.

The operation portion 3 also has a treatment tool insertion port 10 and a suction port 13 in FIG. 3 mounted thereon. A forceps plug 11 is mounted on the treatment tool insertion port 10, and a suction button 12 for performing a suction process is mounted on the suction port 13. The forceps plug 11 is removable from the treatment tool insertion port 10, and the suction button 12 is removable from the suction port 13.

As depicted in FIG. 3, a suction tube 14 has an end connected to the suction port 13 and the other end connected to a suction channel connector 4b. To the suction channel connector 4b, there is connected to an end of a joint tube 15a that is joined to a suction device 15. The other end of the joint tube 15a is joined to the suction device 15 that has a suction pump 15b used as suction means. A channel 16 is used a doubling tube and inserted through the insertion portion 2. The channel 16 is in the form of a tube having a dual use as a fluid channel and a treatment tool insertion channel. The doubling tube 16 extends from within the operation portion 3 that is positioned on the user's hand side of the insertion portion 2 to the distal end portion 5.

The doubling tube 16 includes one end attached in position in fluid communication with an opening 5m defined in the distal end portion 5. The other end of the doubling tube 16 is joined to the suction port 13. A treatment tool tube 17 is held in fluid communication with a “treatment tool inlet hole 10h”. The treatment tool inlet hole 10h is defined in the treatment tool port 10. The treatment tool tube 17 is joined at one end thereof to a middle portion of the doubling tube 16.

A first treatment tool such as a laser knife, a pair of gripping forceps, a pair of biopsy forceps, or the like (i) is inserted into a plug opening 11m in the forceps plug 11, then (ii) extends through the first treatment tool inlet hole 10h, the first treatment tool tube 17, and the doubling tube 16, and (iii) is led into the patient's body from the opening 5m in the distal end portion 5.

When the suction button 12 is operated, a body fluid, dirt, or the like in the lumen is (i) drawn through the opening 5m, the doubling tube 16, the suction port 13, the suction tube 14, the suction channel connector 4b, and the joint tube 15a, and (ii) discharged into a tank, not depicted, disposed in the suction device 15.

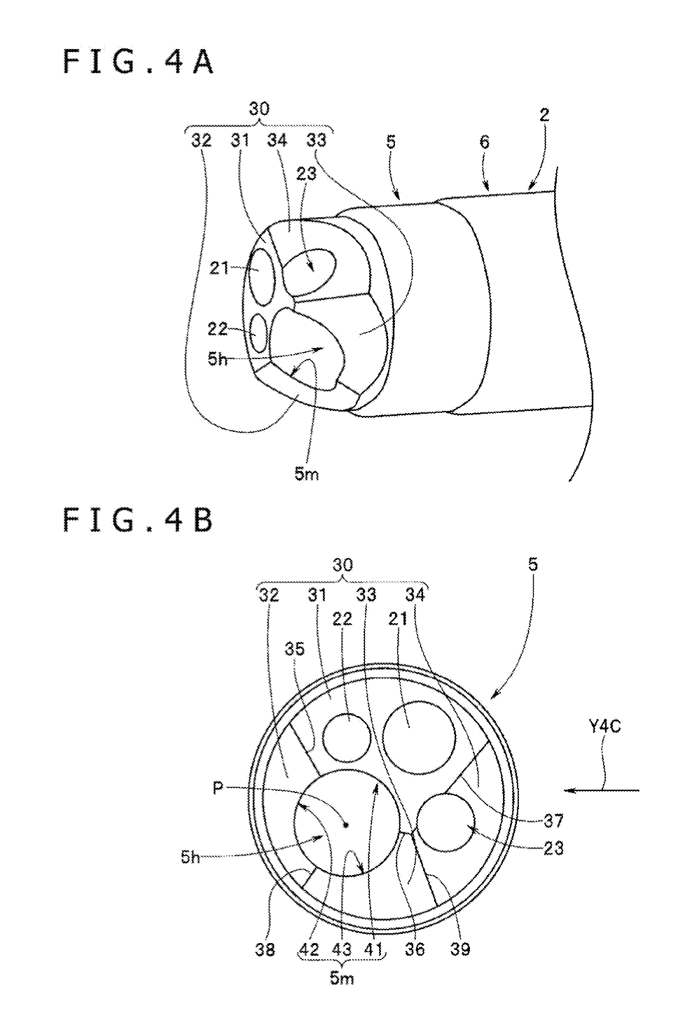

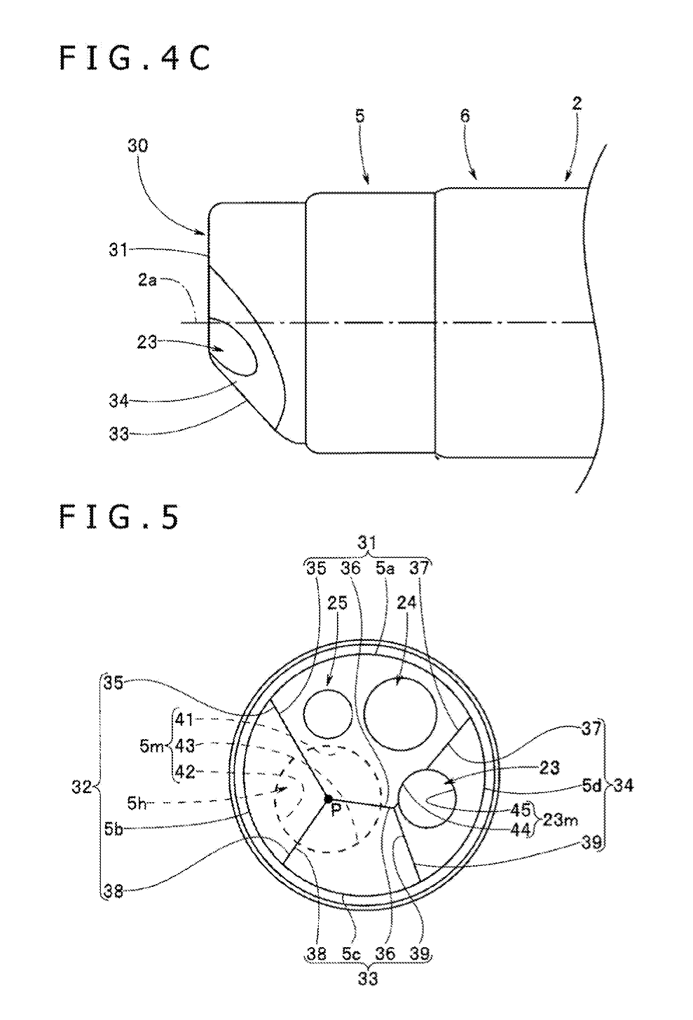

The reference numeral 21 in FIG. 2 represents an objective lens. The structure of the distal end portion 5 will be described hereinafter with reference to FIGS. 4A, 4B, 4C, and 5.

As depicted in FIGS. 4A and 4B, according to the present embodiment, the distal end portion 5 has a distal end face 30 including a first facet 31, a second facet 32 adjacent to the first facet 31, a third facet 33 adjacent to the second facet 32, and a fourth facet 34 adjacent to the third facet 33 and the first facet 31.

In addition to the opening 5m, the distal end portion 5 also has the objective lens 21, an illumination lens 22, and a second through-hole 23. The illumination lens 22 is used as an observation window. The illumination lens 22 is used as an illumination window. The second through-hole 23 is attached to an end of another doubling tube that is different from the doubling tube 16. The opening 5m is used as a distal-end opening of a first through-hole 5h and doubles as a suction opening and a treatment tool outlet. A second distal-end opening 23m has a dual uses as a water delivery opening and a treatment tool outlet from which a slender treatment tool such as a guide wire, a contrast tube, or the like is led out. The doubling tube 16 and the other doubling tube have different inside diameters such that the inside diameter of the other doubling tube is smaller than the inside diameter of the doubling tube 16.

In FIG. 4B, the first facet 31 and the second facet 32 are divided from each other across a first boundary 35. The first facet 31 and the third facet 33 are divided from each other across a second boundary 36. The first facet 31 and the fourth facet 34 are divided from each other across a third boundary 37. The second facet 32 and the third facet 33 are divided from each other across a fourth boundary 38. The third facet 33 and the fourth facet 34 are divided from each other across a fifth boundary 39.

As depicted in FIG. 4C, the first facet 31 is an orthogonal facet that is orthogonal to a longitudinal axis 2a of the insertion portion 2 and provides a foremost face of the insertion portion 2. Although not depicted in FIG. 4C, the second facet 32 is a so-called slanting facet that intersects with the first facet 31 at a predetermined angle. The third facet 33 is a slanting facet that intersects with the second facet 32 at a predetermined angle. The fourth facet 34 is a slanting facet that intersects with the first facet 31 and the third facet 33 at predetermined angles. As a result, the foremost end of the distal end portion 5 is of a tapered shape smaller in diameter than the distal end side of the bendable portion 6. Consequently, the distal end portion 5 of the insertion portion 2 of the endoscope 1 can be pushed into a bile duct or a pancreatic duct which is a narrow lumen and pushed on deeply in the duct. The relationship between the facets 31 through 34 on the distal end face 30 and the through-holes 5h and 23 through 25 will be described hereinafter with reference to FIG. 5.

As depicted in FIG. 5, the first facet 31 is a facet surrounded by a first ridge 5a that is part of the outer periphery of the distal end portion, the first boundary 35, the second boundary 36, and the third boundary 37. The second facet 32 is a facet surrounded by a second ridge 5b that is part of the outer periphery of the distal end portion and positioned adjacent to the first ridge 5a, the fourth boundary 38, and the first boundary 35. The third facet 33 is a facet surrounded by a third ridge 5c that is part of the outer periphery of the distal end portion and adjacent to the second ridge 5b, the fifth boundary 39, the second boundary 36, and the fourth boundary 38. The fourth facet 34 is a facet surrounded by a fourth ridge 5d that is part of the outer periphery of the distal end portion and positioned between the first ridge 5a and the third ridge 5c, the third boundary 37, and the fifth boundary 39.

According to the present embodiment, the first facet 31 has defined therein an objective optical system hole 24 and an illuminating optical system hole 25. The objective optical system hole 24 is used as a third through-hole where observing means such as the objective lens 21 is placed. The illuminating optical system hole 25 is used as a fourth through-hole where illuminating means such as the illumination lens 22 is placed. The second distal-end opening 23m of the second through-hole 23 is defined in both the first facet 31 and the fourth facet 34.

The opening 5m, indicated by the broken line, of the first through-hole 5h is defined so as to be disposed in a position where a point “P” is included in the opening 5m. The point “P” is a point where three boundaries including the first boundary 35, the second boundary 36, and the fourth boundary 38 intersect with each other. In other words, a projected plane of the opening 5m is disposed in a position including the point “P” where the three boundaries intersect with each other.

According to the present embodiment, a projected shape of the opening 5m represents a circle around the point “P”. The opening 5m is defined by joining a first arc 41 formed on the first facet 31, a second arc 42 formed on the second facet 32 and divided from the first arc 41, and a third arc 43 formed on the third facet 33 and divided from the second arc 42 and the first arc 41. In other words, as depicted in FIG. 4B, the distal end face 30 that has the opening 5m includes the first facet 31 including the first arc 41, the second facet 32 including the second arc 42, the third facet 33 including the third arc 43, and the fourth facet 34.

The first facet 31, the second facet 32, the third facet 33, and the fourth facet 34 may be flat facets or curved facets having predetermined curvatures. The first boundary 35, the second boundary 36, the third boundary 37, the fourth boundary 38, and the fifth boundary 39 may be round with predetermined radii. The point “P” may not be at the center of the circle as long as it lies within the projected shape of the opening 5m. It should be noted that either (i) the observing means for observing an examinee or (ii) the illuminating means for applying illuminating light to the examinee may be disposed on the first facet 31, whereas the other means may be disposed on any of the facets 32, 33, and 34 other than the first facet 31. The first through-hole 5h and the second through-hole 23 have respective central axes parallel to the longitudinal axis 2a of the insertion portion 2. The objective optical system hole 24 and the illuminating optical system hole 25 also have respective central axes parallel to the longitudinal axis 2a of the insertion portion 2.

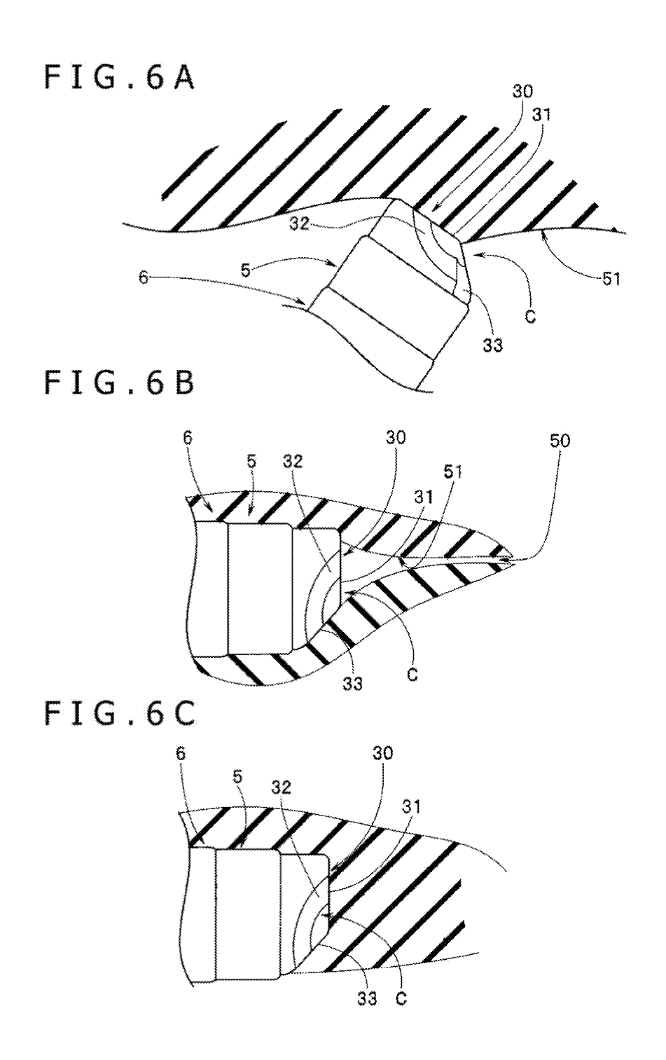

With this arrangement, even when the first facet 31 of the distal end portion 5 is held in close contact with a lumen wall surface 51, as depicted in FIG. 6A, a clearance “C” is created between the lumen wall surface 51 and the second facet 32 and the third facet 33 that are slanting facets. When the distal end portion 5 is pushed and inserted into a narrow lumen 50, spreading the lumen 50, as depicted in FIG. 6B, a clearance “C” is created between the lumen wall surface 51 and the first facet 31 that is an orthogonal facet in front of the latter, even though the second facet 32 and the third facet 33 are held in contact with the lumen wall surface 51. When the first facet 31 and either the second facet 32 or the third facet 33, e.g., the second facet 32, is held in contact with the lumen wall surface 51, as depicted in FIG. 6C, a clearance C is created between the lumen wall surface 51 and the third facet 33.

As described hereinbefore, with the distal end face 30 having (i) the first facet 31 that is an orthogonal facet, (ii) the second facet 32 that is a slanting facet, and (iii) the third facet 33, the opening 5m is defined in the distal end face 30 by (i) the first arc 41 on the first facet 31, (ii) the second arc 42 on the second facet 32, and (iii) the third arc 43 on the third facet 33, the arcs being joined together and the facets being divided from each other. As a consequence, the first facet 31, the second facet 32, and the third facet 33 are unlikely to be covered simultaneously by the lumen wall surface 51. Therefore, the mucous membrane is prevented from being attracted to the distal end face 30 in a suction process.

Since the second distal-end opening 23m of the second through-hole 23 is defined in both the first facet 31 and the fourth facet 34, water that flows out of the second distal-end opening 23m flows along the surface of the first facet 31, removing dirt on the surface of the objective lens 21 or the surface of the illumination lens 22.

When a suction process is carried out while water is flowing out of the second distal-end opening 23m through the opening 5m, the water that has removed dirt from the lens surface is drawn from the opening 5m, and the water flowing out of the second distal-end opening 23m along the fourth facet 34 into the lumen is drawn from the opening 5m. As a consequence, in addition to the surface of the objective lens 21 and the surface of the illumination lens 22, the lumen wall surface 51 is cleaned for good endoscopic observation. In other words, according to the present embodiment, the distal end face 30 can be cleaned even though it has no nozzle.

The ridges of the first arc 41, the second arc 42, and the third arc 43 that define the opening 5m, and the ridges of the fourth arc 44 and the fifth arc 45 that define the second distal-end opening 23m may be round with predetermined radii. The ridge of the arc disposed on the first facet 31 and defining the second distal-end opening 23m may be round with a radius larger than the ridge of the arc on the fourth facet 34. As a result, water flowing out of the second distal-end opening 23m is likely to flow onto the first facet 31, cleaning the first facet 31 well.

The opening 5m in the distal end face 30 may be defined by (i) the first arc 41 on the first facet 31, (ii) the second arc 42 on the second facet 32, (iii) the third arc 43 on the third facet 33, and (iv) a fourth discrete arc, not depicted, on the fourth facet 34, these arcs being joined together. As a result, the first facet 31, the second facet 32, the third facet 33, and the fourth facet 34 are more unlikely to be covered simultaneously by the lumen wall surface 51, so that the mucous membrane is more reliably prevented from being attracted to the distal end face 30 in an suction process.

In the embodiment described hereinbefore, gas delivery may be carried out from the openings 5m and 23m to supply air or carbon dioxide gas to the lumen, expanding the lumen. Suction may be performed through the opening 23m. If air or the like is supplied from the opening 5m, inasmuch as the first facet 31, the second facet 32, and the third facet 33 are unlikely to be covered simultaneously by the lumen wall surface 51, air can reliably be supplied to the inside of the body even when the pressure under which air is ejected from the opening 5m is low.

In sum, one aspect of the disclosed technology is directed to an endoscope comprises an insertion portion that extends in a longitudinal direction thereof. A first facet is disposed on a distal end portion of the insertion portion and is positioned orthogonally to the longitudinal direction. A second facet is disposed on the distal end portion of the insertion portion and is intersected with the first facet adjacent thereto. A third facet is disposed on the distal end portion of the insertion portion and is intersected with the first facet and the second facet adjacent thereto. A fourth facet is disposed on the distal end portion of the insertion portion and is intersected with the first facet and the third facet adjacent thereto. A first opening is held in fluid communication with a distal end of a first channel inserted in the insertion portion in which a projected plane of the first opening at the distal end portion of the insertion portion is disposed in a position including a point where three boundaries between the first facet, the second facet, and the third facet intersect with each other. A second opening is held in fluid communication with a distal end of a second channel inserted in the insertion portion in which a projected plane of the second opening at the distal end portion of the insertion portion is disposed in a position including a boundary across which the first facet and the fourth facet intersect with each other.

The projected plane of the second opening is disposed in a position spaced from a point where three boundaries between the first facet, the third facet, and the fourth facet intersect with each other. A center of the first opening and a center of the second opening are arrayed in a direction substantially parallel to a direction in which the second facet and the fourth facet are arrayed. An objective lens of observing means for observing an examinee and an objective lens of illuminating means for applying illuminating light to the examinee are disposed on the first facet. A center of the objective lens of the observing means and a center of the objective lens of the illuminating means are arrayed in a direction substantially parallel to a direction in which the second facet and the fourth facet are arrayed. An inside diameter of the second opening in the second channel is smaller than an inside diameter of the first opening in the first channel.

An objective lens of observing means for observing an examinee and an objective lens of illuminating means for applying illuminating light to the examinee are disposed on the first facet. An outside diameter of the objective lens of the observing means is larger than an outside diameter of the objective lens of the illuminating means. A first line interconnects a center of the objective lens of the observing means and a center of the first opening in the first channel intersects with a second line interconnects a center of the objective lens of the illuminating means and a center of the second opening in the second channel. The projected plane of the first opening is disposed in a position spaced from a point where three boundaries between the first facet, the third facet, and the fourth facet are intersected with one another. The first channel is connected to suction means at the user's hand side of the insertion portion. A first treatment tool is inserted from the user's hand side of the insertion portion into the first channel. A second treatment tool that is smaller in diameter than the first treatment tool inserted into the first channel is inserted in the second channel. The second channel includes a fluid channel for supplying a fluid forwardly of the distal end portion of the insertion portion.

Another aspect of the disclosed technology is directed an endoscope comprises an insertion portion having a distal end portion that extends in a longitudinal direction. A first channel and a second channel each of which is inserted through the insertion portion. The distal end portion includes a first facet is positioned orthogonally to the longitudinal direction. A second facet is intersected with the first facet adjacent thereto. A third facet is intersected with the first facet and the second facet adjacent thereto. A fourth facet is intersected with the first facet and the third facet adjacent thereto. A first opening is held in fluid communication with a distal end of the first channel in which a projected plane of the first opening at the distal end portion is disposed in a position including a point where three boundaries between the first facet, the second facet, and the third facet are intersected with one another. A second opening is held in fluid communication with a distal end of the second channel in which a projected plane of the second opening at the distal end portion is disposed in a position including a boundary across which the first facet and the fourth facet are intersected with one another.

According to the present disclosure, there is realized an endoscope which prevents the mucous membrane from being attracted thereto in a suction process and which is of excellent manipulability for pushing an insertion portion smoothly into a narrow lumen. The present invention is not limited to only the embodiment described hereinbefore, but various changes and modifications may be made without departing from the scope of the invention.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An endoscope comprising:
an insertion portion extends in a longitudinal direction thereof;
a first facet disposed on a distal end portion of the insertion portion and positioned orthogonally to the longitudinal direction;
a second facet disposed on the distal end portion of the insertion portion and being intersected with the first facet adjacent thereto;
a third facet disposed on the distal end portion of the insertion portion and being intersected with the first facet and the second facet adjacent thereto;
a fourth facet disposed on the distal end portion of the insertion portion and being intersected with the first facet and the third facet adjacent thereto;
a first opening held in fluid communication with a distal end of a first channel inserted in the insertion portion in which a projected plane of the first opening at the distal end portion of the insertion portion is disposed in a position including a point where three boundaries between the first facet, the second facet, and the third facet being intersected with one another; and
a second opening being held in fluid communication with a distal end of a second channel inserted in the insertion portion in which a projected plane of the second opening at the distal end portion of the insertion portion is disposed in a position including a boundary across which the first facet and the fourth facet being intersected with one another.

2. The endoscope of claim 1, wherein the projected plane of the second opening is disposed in a position spaced from a point where three boundaries between the first facet, the third facet, and the fourth facet are intersected with one another.

3. The endoscope of claim 1, wherein a center of the first opening and a center of the second opening are arrayed in a direction substantially parallel to a direction in which the second facet and the fourth facet are arrayed.

4. The endoscope of claim 1, wherein an objective lens of observing means for observing an examinee and an objective lens of illuminating means for applying illuminating light to the examinee are disposed on the first facet.

5. The endoscope of claim 4, wherein a center of the objective lens of the observing means and a center of the objective lens of the illuminating means are arrayed in a direction substantially parallel to a direction in which the second facet and the fourth facet are arrayed.

6. The endoscope of claim 1, wherein an inside diameter of the second opening in the second channel is smaller than an inside diameter of the first opening in the first channel.

7. The endoscope of claim 6, wherein an objective lens of observing means for observing an examinee and an objective lens of illuminating means for applying illuminating light to the examinee are disposed on the first facet; and
an outside diameter of the objective lens of the observing means is larger than an outside diameter of the objective lens of the illuminating means.

8. The endoscope of claim 7, wherein a first line interconnects a center of the objective lens of the observing means and a center of the first opening in the first channel intersects with a second line interconnects a center of the objective lens of the illuminating means and a center of the second opening in the second channel.

9. The endoscope of claim 1, wherein the projected plane of the first opening is disposed in a position spaced from a point where three boundaries between the first facet, the third facet, and the fourth facet are intersected with one another.

10. The endoscope of claim 1, wherein the first channel is connected to suction means at the user's hand side of the insertion portion.

11. The endoscope of claim 1, wherein a first treatment tool is inserted from the user's hand side of the insertion portion into the first channel.

12. The endoscope of claim 11, wherein a second treatment tool which is smaller in diameter than the first treatment tool inserted into the first channel is inserted in the second channel.

13. The endoscope of claim 1, wherein the second channel includes a fluid channel for supplying a fluid forwardly of the distal end portion of the insertion portion.

14. An endoscope comprising:
an insertion portion having a distal end portion that extends in a longitudinal direction, a first channel and a second channel each of which being inserted through the insertion portion wherein
the distal end portion includes:
a first facet positions orthogonally to the longitudinal direction;
a second facet intersects with the first facet adjacent thereto;
a third facet intersects with the first facet and the second facet adjacent thereto;
a fourth facet intersects with the first facet and the third facet adjacent thereto;
a first opening being held in fluid communication with a distal end of the first channel in which a projected plane of the first opening at the distal end portion is disposed in a position including a point where three boundaries between the first facet, the second facet, and the third facet being intersected with one another; and
a second opening held in fluid communication with a distal end of the second channel in which a projected plane of the second opening at the distal end portion is disposed in a position including a boundary across which the first facet and the fourth facet being intersect with one another.

15. The endoscope of claim 14, wherein the projected plane of the second opening is disposed in a position spaced from a point where three boundaries between the first facet, the third facet, and the fourth facet are intersected with one another.

16. The endoscope of claim 14, wherein an inside diameter of the second opening is smaller than an inside diameter of the first opening in the first channel.

17. The endoscope of claim 16 further comprising:
a first objective lens for observing an examinee disposed on the first facet;
a second objective lens for applying illuminating light to the examinee disposed on the first facet; wherein an outside diameter of the first objective lens is larger than an outside diameter of the second objective lens.

18. The endoscope of claim 17, wherein a first line interconnecting a center of the first objective lens and a center of the first opening intersects with a second line interconnecting a center of the second objective lens and a center of the second opening in the projected plane.

19. The endoscope of claim 14, wherein the projected plane of the first opening is disposed in a position spaced from a point where three boundaries between the first facet, the third facet, and the fourth facet are intersected with one another.
